SEQUENCE LISTING (2 sequences)

SEQ ID NO: 1 - DNA, 503 bp, Human immunodeficiency virus
gtgctggaggagatgtccctgcccgggcgctggaagcccaagatgatcggcggcatcggcggcttcatcaaggtccgccagtacgaccagatcctgatcgagatctgcggccacaaggccatcggcaccgtgctggtgggacccacccccgtgaacatcatcggccgcaacctgctgacccagatcggctgcaccctgaacttccccatctcccccatcgagaccgtgcccgtgaagctgaagcccggcatggacggccccaaggtgaagcagtggcccctgaccgaggagaagatcaaggccctggtcgaaatctgcaccgagatggagaaggagggcaaaatctccaagatcggccccgagaacccctacaacacccccgtgttcgccatcaagaagaaggactccaccaagtggcgcaagctggtggacttccgcgagctgaacaagcgcacccaggacttctgggaggtccagctgggcatcccccaccccgccggcct

SEQ ID NO: 2 - protein, 168 aa, Human immunodeficiency virus
VLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGL

(19) United States
(12) Patent Application Publication
(10) Pub. No.: US 2003/0228327 A1
(43) Pub. Date: Dec. 11, 2003

(54) DNA-based plasmid formulations and vaccines and prophylactics containing the same

(76) Inventors: Alfred W. Lasher, (US); Joseph D. Kittle JR., (US); Steven G. Widen, (US)

Correspondence Address: LOCKE LIDDELL & SAPP LLP, 600 TRAVIS, 3400 CHASE TOWER, HOUSTON, TX, 77002-3095, US

(21) Appl. No.: 10/288,251

(22) Filed: Nov. 4, 2002

Related U.S. Application Data
(60) Provisional application No. 60/337,860, filed on Nov. 5, 2001.

(51) Int. Cl. A61K 39/21; A61K 48/00

(52) U.S. Cl. 424/188.1; 424/93.21; 424/208.1

(57) ABSTRACT

The invention is a general method for improving the performance of the DNA-based vaccines. The method utilizes a complex DNA-generated profile of antigens to extend the effects of DNA-based vaccines and to broaden the immune response. This broadened immune response in turn improves the protection of the recipient from divergent (but related) strains of a pathogen. In addition, it effectively improves the efficacy of DNA-based vaccines used for treatment of viral diseases, including acquired immunity disorder (AIDS). One embodiment, where the target viral pathogen is HIV (the causative agent for aids), the method identifies an orderly set of plasmids of related sequences that may be used to prime a broad and strong immune response to HLA-restricted viral antigens. This mixture of plasmids is thus capable of priming an appropriate immune response to reduce the viral burden in HIV infected patients or to protect uninfected patients from HIV infection.

### FIELD OF THE INVENTION

This invention relates to the field of vaccines and disease prophylaxis, more particularly to vaccine and prophylactic formulations having multiple forms of one or more antigens.

### BACKGROUND OF THE INVENTION

While vaccinations are one of the most cost-effective medical methods for saving lives, they have not been effectively developed for many of the most serious human diseases, including pneumonia caused by Streptococcus pneumoniae, diarrhea caused by rotavirus, and Shigella. There is an increasing need to develop effective vaccines and prophylactics as is evident with the increasing HIV, tuberculosis and malaria epidemics as well as other parasitic diseases and antibiotic-resistant bacteria, yet this development has proven difficult for many pathogens. For example, the influenza virus is notorious for antigenic drift so that new vaccines are constantly being developed; research efforts continue in attempts to identify effective vaccines for rabies (Xiang, et al, 1994), herpes (Rouse, 1995); tuberculosis (Lowrie, et al, 1994); HIV (Coney, et al, 1994) as well as many other diseases of importance in developed and undeveloped countries.

Furthermore, many bacterial strains once easily treated by conventional antibiotics are now becoming increasingly resistant to treatment. Standard polyvalent vaccines are typically designed to act against a limited number of defined pathogenic strains. There is no general method for engineering vaccines or prophylactics that are effective against classes of pathogens that exhibit different of varying antigens.

Many currently used vaccines are composed of live/attenuated pathogens (Ada, 1991) which, when inoculated, infect cells and elicit a broad immune response in the host. Live vaccines are often superior to antigen or subunit vaccines because of their tendency to elicit a broad level protective response. However, serious disadvantages in using such vaccines include vaccine-induced infection, problems with producing and storing the vaccine and failure to engender any immune response (i.e., where antigen presentation is limited). Perhaps the most troubling aspect of using live vaccines is the propensity for actually causing the disease against which protection is intended. Experience with some of the polio and measles vaccines has demonstrated that this may be a serious risk.

An alternative to using live/attenuated pathogen vaccines is the use of single proteins or a limited number of proteins associated with the pathogen to evoke an immune response. However, there is no assurance that antibodies produced in response to an antigen will provide protection against the pathogen providing the antigen. Therefore, it may be necessary to test a large number of antigens isolated from a pathogen. It is possible no single antigen will prove effective as a vaccine because the ability of subunit or killed vaccine preparations to elicit a broad immune response is generally quite limited. In particular, peptide subunit vaccines have little effectiveness in evoking a killer T-cell mediated response.

These disadvantages of conventional vaccines can be overcome by using what is called “genetic immunization” (Tang, 1992). This technology involves inoculating simple, naked plasmid DNA encoding a pathogen protein (antigen) into the cells of the host. The antigen is produced intracellularly and, depending on the attached targeting signals, can result in effective major histocompatibility complex (MHC) class I or II presentation (Ulmer, et al, 1993; Wang, et al, 1993). The risk of infection is essentially eliminated and the DNA can be delivered to cells not normally infected by the pathogen. Compared to conventional vaccines, the production of genetic vaccines is straightforward and DNA is considerably more stable than proteinaceous or live/attenuated vaccines.

Genetic immunization (a.k.a. DNA, polynucleotide, etc. immunization) with specific genes has shown promise in several model systems of pathogenic disease (Davis, et al, 1993; Conry, et al, 1994; Xiang, et al, 1994), and a few natural systems (Cox, et al, 1993; Fynan, et al, 1993). The use of DNA (or RNA) thus overcomes some of the problems encountered when an animal is presented directly with an antigen.

For development of DNA vaccine, a polypeptide-encoding region of the pathogen genome is selected as the parental antigen. Thus, for any particular antigenic polypeptide typically one or two regions of the antigen (approximately nine amino acid long and called the “dominant epitope”) are presented and dominate the MHC mediated immune response.

Despite promising initial results with genetic vaccination, there remains the basic problem of identifying the particular gene or genes that will express an antigen capable of priming the immune system for rapid and protective response to pathogen challenge. One solution proposed to solve this problem is to prepare a DNA vaccine that contains multiple plasmids each encoding a separate portion of the pathogens genome (Johnston S A, Barry M A. Vaccine Jun. 15, 1997; (8): 808-809). In the case of well-studied pathogens, such as HIV, the dominant epitope of many of the antigen proteins have been mapped. Due to a high mutation rate inherent to the retroviral replication mechanism and the extreme selective pressure that occurs during a persistent infection, the viral population driving the AIDS pandemic is highly variable. This variability appears to limit the effectiveness of DNA-based vaccines in two ways. First, there are many variants present in the population that can be transmitted to currently uninfected hosts, and the vaccine would need to be able to suppress an unpredictable and perhaps previously undetected strain of the virus. Second, in the case of a persistent infection, the virus population within the patient can undergo an antigenic shift and be dominated by a novel variant of the infecting virus(es).

The method presented here for broadening vaccine/prophylactic specificity is distinct from previous approaches which use a large number of plasmids encoding unrelated antigens from a pathogen. This may be accomplished, for example, by mixing plasmids which contain genes from different regions for the pathogen's genome in the hopes of triggering a sufficient immune response to one of the antigenic regions.

### SUMMARY OF THE INVENTION

The present invention relates to a composition comprising multiple nucleic acid sequences encoding protein or peptide antigens from one or more pathogenic organisms. It provides methods and compositions for inducing an enhanced immune system response in host organisms. Examples of such pathogens include, but are not limited to, the retroviral pathogen HIV, which is responsible for the depletion of the immune system observed in Acquired Immune Deficiency Syndrome (AIDS).

### BRIEF DESCRIPTION OF THE DRAWINGS

In order to more fully understand the drawings referred to in the detailed description of the present invention, a brief description of each drawing is presented, in which:

FIG. 1 depicts the nucleic acid sequence of the parental peptide from Pol HTLV-IIIb. Two sites of multivariance are shown in bold print.

FIG. 2A shows the amino acid sequence of the parental antigenic peptide from Pol HTLV-IIIb. The multivariant epitopes are shown in bold. In FIG. 2B, amino acid substitutions within the multivalent epitopes are shown. These are designed from known sequences of clinical isolates of HIV. All possible combinations of the substitutions in the two epitopes are used in the design of the vaccine. The actual antigen is fused to a peptide for expression of the antigen from a DNA vaccine.

### DETAILED DESCRIPTION OF THE INVENTION

As used herein, the following terms shall have the designated meanings:

Hyperpolyvalent—a preparation where twenty or more distinct plasmids are used as components in a DNA vaccine, and are used to evoke a host immune response that, while effective against a target pathogen, is broader that can be invoked by any of the plasmids delivered singly.

Nucleic acid sequence—a polynucleotide chain that consists of various combinations of four monomers: adenine, guanine, cytosine, and thymine.

Full-length nucleic acid sequence—a nucleic acid sequence that encodes a protein.

Fragment of nucleic acid sequence—a portion of a full-length nucleic acid sequence that only encodes a region of a protein.

Antigen—the substance that stimulates the immune system to produce antibodies, usually a protein.

Mutation—a nucleotide within a wild-type gene was substituted with a different nucleotide.

Conservative substitution—Conservative amino acid substitutions refer to the interchangeability of residues having similar side chains. For example, a group of amino acids having aliphatic side chains is glycine, alanine, valine, leucine and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; a group of amino acids having sulfur-containing side chains is cysteine and methionine. Preferred conservative amino acid substitutions are: valine-isoleucine-leucine; phenylalanine-tyrosine; lysine-arginine; alanine-valine and asparagine-glutamine.

Epitope—the portion of an antigen that is recognized by and combines with the antibody.

Dominant epitope—an epitope that is critical to the interaction between antibody and antigen.

The improvement is a hyperpolyvalent mixture of nucleic acid sequences that results in the expression of a mixture of related but distinct antigens within the host organism. These can include DNA, RNA, nucleic acids other than these, and both full-length and partial nucleic acid sequences. The nucleic acids may be presented as “naked” nucleic acid, or in a vector. Such vectors would include but are not limited to self-replicating plasmids and virus vectors (e.g. adenovirus). The term plasmid is used to refer to any small, independently-replicating piece of DNA that can be transferred from one organism to another. These include linear or circular DNA molecules that are found in both pro- and eucaryotes. Plasmids are capable of being integrated into the hosts genome or remaining independent and can be used to transfer genes to a host. This improvement uses a large number of closely related plasmid variants to immunize the host immune system so it can mount a response to a challenge by a variety of related but distinct strains of the target pathogens. This set of plasmids has a systematic design that defines the range of epitopes used to prime the host immune system. The present method uses polynucleotides derived from one or a small number of genes in the target pathogen, and multiple variants of the selected antigen are generated.

DNA vaccines are particularly effective for evoking a cytolytic T lymphocyte (CTL) response. The specificity of CTL and other immune responses are dependent in detail on the types and amounts of antigen produced and on the presentation of antigens on cells that receive plasmids. Using a single plasmid encoding an antigen to immunize an animal will promote the maturation of T cells specific for that antigen. However, the overall response may be dominated by a highly specific reaction and many components of the overall immune response will be relatively intolerant of even minor antigenic variation. A mixture of plasmids that expresses a set of closely related polypeptides but no single dominant specific antigen would lead to an immune response that is broad in its specificity. The set of plasmids is developed to vary the nature of the epitopes presented and the relative efficiency of presentation results in a broad specificity toward the pathogen and variants. This allows the host to develop effective resistance to the pathogen, in the face of challenge by previously undescribed variant strains or to overcome rapid mutation of the pathogen in response to immune system elimination.

Development of an Orderly Set of Plasmids as Components of a Vaccine.

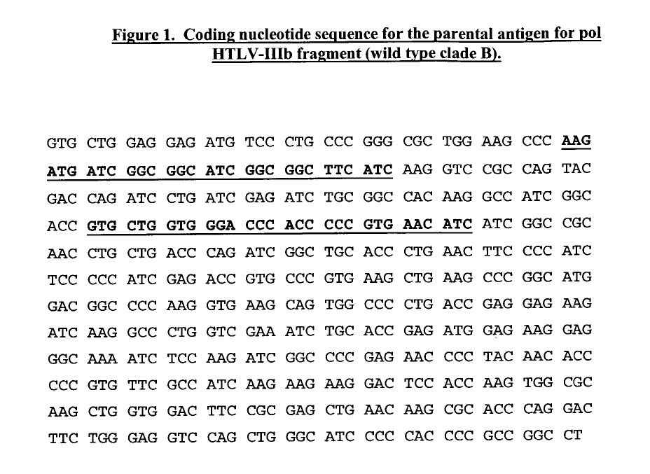

In one aspect, for development of DNA vaccine, a polypeptide-encoding region of the pathogen genome is selected as the parental antigen. As an example, the nucleotide coding sequence for the parental antigen of the HIV gene pol is provided in FIG. 1. However, this is only given as an example and is not meant to limit the scope of this invention to HIV alone nor is it meant to limit this invention to the pol gene of HIV. For any particular antigenic polypeptide, typically one or two regions of the antigen (approximately nine amino acid long and called the “dominant epitope”) are presented and dominate the MHC mediated immune response. The DNA polynucleotide encoding each amino acid of the antigenic region can be fused to a polynucleotide that encodes an antigenic peptide of the type typically used to enhance the immunicity of the resulting fused polypeptide.

In the present invention, a set of plasmids are created containing nucleic acid base sequences that, when translated, systematically replace amino acid residues in the dominant epitope region, for example, by substituting with conservative amino acid replacements. Examples of conservative changes can be identified from Table 1 below.

TABLE 1

| Amino acid | A G P C M K |
|---|---|
| Conservative | G P G M C A |
| replacement(s) | |
| | V A S T T E |
| | S S R |
| | D |

The amino acids are designated by their standard one letter code.

However, random mutations may also be incorporated. Furthermore, the amino acid sequence flanking the epitope can be varied to alter the strength and specificity of the MHC restriction system's presentation of the antigen.

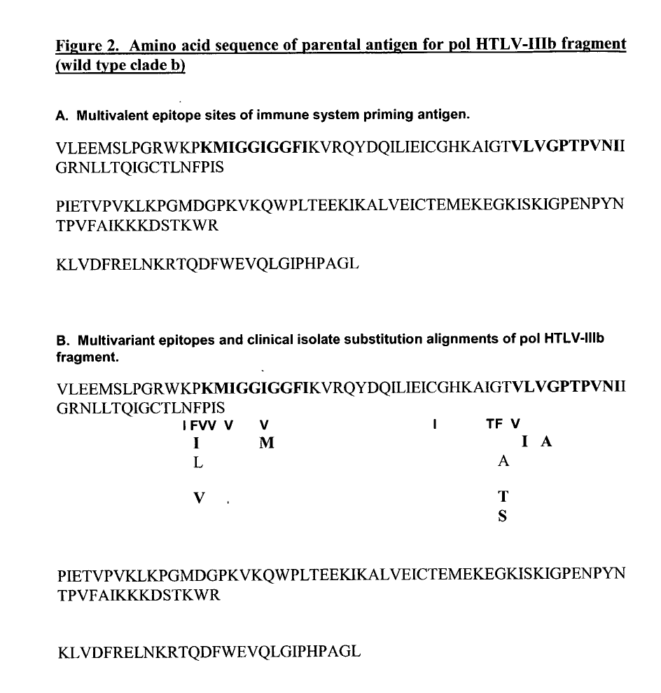

An example of a translated polynucleotide sequence with dominant epitopes is shown in FIGS. 2A and 2B. An example of multivariant epitopes using a fragment of the HIV pol gene is shown in FIG. 2B. However, this is only given as an example and is not meant to limit the scope of this invention to HIV alone nor is it meant to limit this invention to the pol gene of HIV. Additionally, the hyperpolyvalent mixture can include the variants for one or several genes of the same organism.

One key feature of the system is to create a mixture of plasmids encoding a rich variety of related antigens, each capable of expressing significant amounts of antigen in the host cells that receive it. The variant dominant epitopes presented in each transfected host cell play a role in triggering the host organism's total immune response. T lymphocytes that can key in on a broader range of the available epitopes are favored in selection of those with restrictively narrow response specificity. The overall CTL response is thus dominated by T lymphocytes with immune response profiles that are broader than the response triggered by a single antigenic plasmid.

The compositions of the present invention can be used for vaccination of animals against a variety of diseases caused by bacterial and/or viral pathogens. For example, the compositions of the present invention can be used for vaccination against HIV, rabies, plague, influenza, smallpox, Ebola, Marburg, and West Nile viruses as well as treatment for infection with anthrax, resistant strains of staphylococcus and streptococcus, Salmonella, E. coli, and other bacterial agents. This technology can also be used in treatment of different cancers or toxins. Additionally it can be used to strengthen current antibiotics, vaccines, or prophylactics. In a current embodiment (example) the compositions of the present invention can be used to vaccinate animals against HIV.

Testing as a Part of Vaccine Development

This aspect of the invention is tested in a mouse model system, a step for testing any vaccine prior to assessment in human volunteers. The general concept of testing DNA based vaccines in mouse model systems is well known to those skilled in the art of vaccine development. In such a test, a suitable strain of mice is inoculated using the plasmid based vaccine formulation. This formulation is delivered using standard techniques of injecting naked DNA intramuscularly. The delivery system may be saline solution of a gel-based depo for gradual release of plasmid. Adjuvants may be used to enhance the immune response to the antigen expressed from the DNA plasmids. Cells that receive and express the plasmid mixture present processed a mixture of related antigens to the immune system. Supplying the cytokine IL6 along with the plasmid may further stimulate the immune system. While IL6 may be supplied as a protein, for purposes of this test the cytokine is encoded on as an expressible DNA sequence inserted into a plasmid, so that cells in the vicinity of injection site produce the cytokine. The immunization is boosted by periodic re-inoculation with the vaccine formulation to enhance the immune response. Control mice are injected with saline alone and additional control mice are inoculated with IL6 plus adjuvant only. A third control group consists of mice inoculated with a single species of antigen supplying plasmid, which stimulates a relatively narrow immune response to the specific HIV (pol) antigen presented. The mice are sacrificed periodically (after 2, 6 and 12 weeks) and spleens are collected to perform a test of the CTL response. The standard test is to look for the activation of the CTL responses using synthetic peptides (Wu L, Barry M A., Mol Ther Sep. 2, 2000; (3): 288-297). In this test, one specific variety of dominant epitope is deliberately omitted form the plasmid mixture used to inoculate the test mice. The parental antigen is omitted so that the known dominant antigen may be used to assay the effectiveness of the vaccine in stimulating a broad CTL response. The reasoning is that the broad CTL response stimulated by the complex plasmid mixture will be able to recognize a related epitope not specifically supplied in the vaccine (in comparison to the control mice). Furthermore, the assay for the parental type is highly reliable. Additional antigens are also tested to verify the effectiveness of the immune response to a broad range of antigens.

Practical creation of large sets of sequence related DNA plasmids is possible using recently described automated DNA synthesis techniques (U.S. Ser. No. 60/276,161). These techniques use automated liquid handling equipment to generate tens to hundreds of sequence related polynucleotides for cloning. Such a system is used to create an orderly set of clones with the engineered changes to the antigen's dominant epitope and flanking regions.

An important aspect of this invention is a method for the vaccination against HIV, the causative agent for AIDS. The current treatments for HIV/AIDS are problematic due to the antigenic shift/mutation rate of the virus. By vaccination with variants of the genes/pathogenic epitopes contained in HIV, a broader immune response is triggered. This would help defend the host against the antigenic shift observed with HIV.

The present invention provides a pharmaceutical product, comprising of a hypervalent mixture of the gene or genes of interest as part of a plasmid. This hypervalent mixture includes epitope variants of each of the gene or genes of interest. The number of variants is at least nine; one per amino acid within the dominant epitope. Each polynucleotide operatively codes for a biologically active polypeptide. The hypervalent polynucleotide is administered in a physiologically acceptable form, in a container, and a notice associated with the container in form prescribed by a governmental regulatory agency regulating the manufacture, sale, and/or use of pharmaceuticals. Obviously many modifications and variations of this invention are possible in the light of the above teachings. It is therefore to be understood that within the scope of the appended claims the invention may be practiced otherwise than as specifically described.

The invention is further described by the following examples, which are provided for illustrative purposes only and are not intended nor should they be construed as limiting the invention in any manner. Those skilled in the art will appreciate that variations on the following examples can be made without deviating from the spirit or scope of the invention.

### EXAMPLE I

The mouse strain BALB/C (H-2d) is used to demonstrate the effect of the hyperpolyvalent vaccination on ct cell response. In brief, thirty mice are divided into three groups of ten animals each. The mice are bred and housed in a pathogen free environment. The three groups are: control group of mice injected only with saline, a group of mice injected with a single plasmid type with the parental sequence, encoding the target epitope and a third group injected with the hypervalent vaccine which is formulated to encode a mixture of sequence types but deliberately not containing the parental epitope. This formulation is used so that any response to the parental epitope is a direct indication of broad specificity of t-cell response. The animals are injected (50 microliters of saline solution) with purified DNA plasmid (vaccine or control) into the Quadriceps and tibialis muscles using a 0.5 cc syringe with at 28-gauge needle. Spleens and blood serum are collected from animals sacrificed at 2, 4 and 12-week intervals. Western Blot Assays are used to measure humeral (antibody response) against viral encoded antigens and CTL response is measured using the T-cell activation assays (Ulmer, J. B., et al, Science 259:1745-1749).

From the foregoing, it will be observed that numerous variations and modifications may be effected without departing from the true spirit and scope of the novel concepts of the invention.

What is claimed is:

1. A polymorphic vaccine comprising a hypervalent mixture of nucleic acid sequences capable of expressing a mixture of distinct antigens within the host organism; the host immune response being broader than that invoked by any one member of the hypervalent mixture by itself.

2. The vaccine of claim 1, wherein the hypervalent mixture of nucleic acid sequences are self-replicating plasmids and virus vectors.

3. The vaccine of claim 2, wherein the self-replicating plasmids and virus vectors are adenovirus.

4. The vaccine of claim 1, wherein the hypervalent mixture of nucleic acid sequences are in a vector.

5. A DNA based vaccine or prophylactic comprising twenty or more distinct plasmids, each of the plasmids having a nucleotide-encoding region for a distinct antigen of a pathogen and furthermore wherein none of the plasmids is dominant and wherein said plasmids are capable of evoking a broad specificity toward the pathogen; the host immune response being broader than that invoked by any of the plasmids individually.

6. The vaccine of claim 5, wherein the pathogen is retroviral pathogen HIV.

7. The vaccine of claim 5, wherein at least one of the plasmids is a linear or circular DNA molecule from a prokaryotic cell or an eukaryotic cell.

8. The vaccine of claim 5, wherein the plasmids or variants thereof are capable of immunizing the host immune system in order to mount a response to a challenge by the target pathogen.

9. The vaccine of claim 5, wherein each of the plasmids contain epitopes capable of priming the host immune system.

10. The vaccine of claim 5, wherein the nucleic acid sequences are polynucleotides derived from at least one gene in the target pathogen such that multiple variants of the selected antigens are generated.

11. A polymorphic vaccine against at least one pathogen, wherein said vaccine comprises a mixture of distinct plasmids, each of the plasmids having a nucleotide-encoding or polypeptide-encoding region for expression of related but distinct antigens, wherein none of the plasmids is dominant and further wherein said plasmids are capable of evoking a broad specificity toward the pathogen and variants thereof; the host immune response being broader than that invoked by any of the plasmids singularly.

12. The polymorphic vaccine of claim 11, wherein the distinct plasmids are composed of variants of the parental plasmid or the combination of parental plasmid and variants thereof.

13. The polymorphic vaccine of claim 11, wherein the plasmids, when expressed, are capable of generating a polypeptide for either the parental epitope or a variant thereof.

14. The polymorphic vaccine of claim 11, wherein the vaccine is for HIV, rabies, plague, influenza, smallpox, Ebola, Marburg, West Nile virus, anthrax, resistant strains of staphylococcus and streptococcus, Salmonella or E. Coli or any combination thereof.

15. The polymorphic vaccine of claim 11, wherein the pathogen is a bacteria or virus.

16. The polymorphic vaccine of claim 11, wherein the vaccine is for the treatment of cancer or toxins.

17. The polymorphic vaccine of claim 11, wherein pathogen is retroviral pathogen HIV.

18. The polymorphic vaccine of claim 12, wherein the plasmids are capable of encoding for pathogenic variants by conservative substitution of the amino acid residues of the parental epitope.

19. The polymorphic vaccine of claim 1, wherein the nucleic acid sequences include AAG ATG ATC GGC GGC ATC GGC GGC TTC ATC.

20. The polymorphic vaccine of claim 1, wherein the nucleic acid sequences Include GTG CTG GTG GGA CCC ACC CCC GTG AAC ATC.
